(19) United States
(12) Patent Application Publication
(10) Pub. No.: US 2024/0407531 A1
(43) Pub. Date: Dec. 12, 2024

(54) Dual Cosmetic Product

(71) Applicant: Blancour, Inc., Seal Beach, CA (US)

(72) Inventors: Ariel Young Tanoto, Cambridge, MA (US); Rebecca Nicole Gomez, Seal Beach, CA (US)

(21) Appl. No.: 18/735,279

(22) Filed: Jun. 6, 2024

Related U.S. Application Data
(60) Provisional application No. 63/506,877, filed on Jun. 8, 2023.

(51) Int. Cl. A45D 40/02; A45D 40/24; A61K 8/02

(52) U.S. Cl. CPC A45D 40/02; A45D 40/24; A61K 8/0229; A61K 2800/87; A61K 2800/88

(57) ABSTRACT

A cosmetics product includes compositions in a cosmetically-acceptable medium which form a solid cosmetic housed in a dispenser. The solid cosmetic includes a first cosmetic composition, including a first pigment, and a second cosmetic composition. The second cosmetic composition is either colorless or is differentiated from the first cosmetic composition by one or more of color, pigment, or finish. The solid composition is in the form of a stick. Along the width of the top of the stick, the depth of the first portion gradually decreases from a larger depth to a smaller depth. The smallest depth of the first portion can be a point, rounded, square, or any another shape. In some implementations, in a cross-section along a plane that is perpendicular to an axis X along a length of the solid composition, a boundary between the first and second blocks has a “V” shape.

### CROSS-REFERENCE TO RELATED APPLICATIONS

This application is a non-provisional application that claims priority to, and the benefit of, prior filed U.S. provisional patent application Ser. No. 63/506,877, entitled “Dual Cosmetic Product”, filed Jun. 8, 2023, and hereby incorporated by reference.

### BACKGROUND

Conventional cosmetics, such as contours, typically use many individual compounds with separate bottles, containers, or applicators.

For example, cream contours typically are available in a stick form, which is cylindrical in shape and highly pigmented. Such sticks typically are provided in a container with a manual twisting mechanism, typically on the bottom of the container, which pushes up the stick. An individual applies the contour in a streak, typically using only one color, to shadow parts of the face, and then blends the contour out using a brush or sponge. Sometimes the container for the stick includes a built-in buffing brush in the body of the packaging.

As another example, powder contours typically are provided in a container called a compact, in powder form. The compact may include an assortment of dark to light shades across a palette of a given hue. Such palettes are meant to allow the individual to mix or match for greater precision in the intensity of contour. Such powder contours typically are applied with a brush. The individual may use different powders of different pigments on different parts of the face, and blend them using a brush or sponge.

### SUMMARY

This Summary introduces a selection of concepts in simplified form that are described further below in the Detailed Description. This Summary neither identifies key or essential features, nor limits the scope, of the claimed subject matter.

A cosmetics product includes compositions in a cosmetically-acceptable medium which form a solid cosmetic housed in a dispenser. The solid cosmetic includes a first cosmetic composition, including a first pigment, and a second cosmetic composition. The second cosmetic composition is either colorless or is differentiated from the first cosmetic composition by one or more of color, pigment, or finish. The solid composition is in the form of a stick. Along the width of the top of the stick, the depth of the first portion gradually decreases from a larger depth to a smaller depth. The gradual decrease can be linear and monotonic. The smallest depth of the first portion can be a point, can be rounded, can be square, or can be any another shape. In some implementations, in a cross-section along a plane that is perpendicular to an axis X along a length of the solid composition, a boundary between the first and second blocks has a “V” shape, with two straight lines extending from a point at an angle to each other.

The following Detailed Description references the accompanying drawings which form a part of this application, and which show, by way of illustration, specific example implementations. Other implementations may be made without departing from the scope of the disclosure.

### BRIEF DESCRIPTION OF THE DRAWINGS

FIGS. 1A to 1F illustrate example constructions of a combined cosmetic.

FIG. 2 includes views of example application techniques.

### DETAILED DESCRIPTION

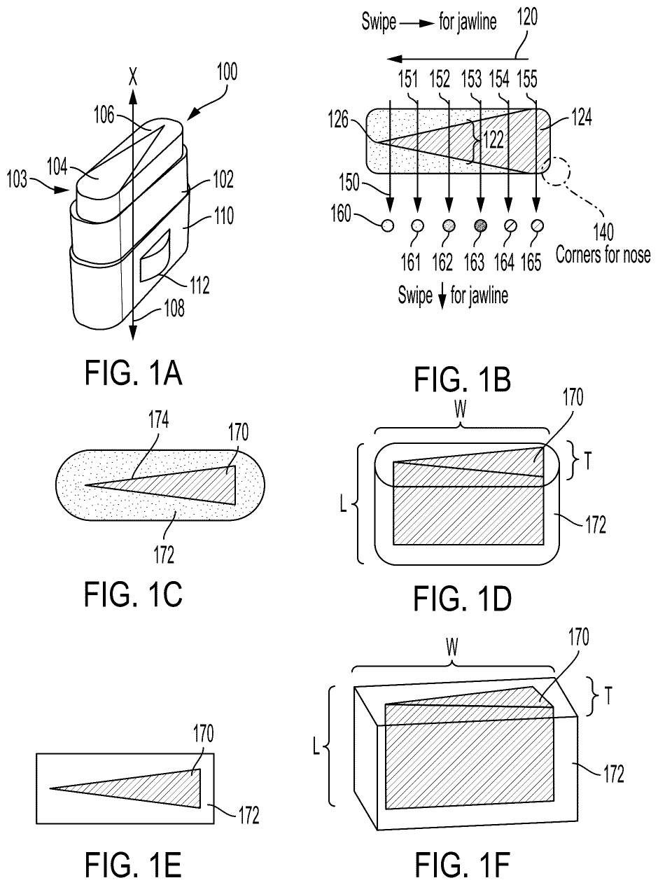

FIGS. 1A through 1F are illustrations of embodiments of a cosmetic product. As shown in FIG. 1A, the cosmetics product includes one or more compositions in a cosmetically-acceptable medium which form a solid cosmetic 100. The solid cosmetic can be housed in a dispenser 102. The term “solid cosmetic” means a cosmetic having the property of lacking the ability to flow under the action of its own weight, under normal storage conditions. The term “cosmetically-acceptable medium” means a medium that is not toxic, is suitable for application to the keratinous outer surfaces of bodies of human beings, such as hair, nails, and outer layer of skin, and forms a flexible film adapted to the nature of the outer surface to which the medium is applied, resulting in comfortable and extended wear.

The solid cosmetic includes a first cosmetic composition 104 and a second cosmetic composition 106. The first cosmetic composition includes a first pigment. The second cosmetic composition is either colorless or is differentiated from the first cosmetic composition by one or more of color, pigment, or finish. The solid composition is in the form of a stick. The term “stick” is used to designate a composition in solid form, generally in the form of an elongated block, enabling the cosmetic to be transferred by friction.

As shown in FIGS. 1A to 1F, the solid cosmetic can include at least two distinct portions, each portion having a respective formulation different from the formulation of the other portions. As an example, one portion can be a sheer tinted contour formulas, the other portion can be clear or colorless. A first portion can be in the form of a first block or stick. The second portion also can be in the form of a second block or stick which mates, within an overall shape of the solid composition, with the first block of stick. The first and second blocks may extend over the entire length (L, FIGS. 1D and 1F) of a stick and vary in composition along the width (W, FIGS. 1D and 1F) and depth (T, FIGS. 1D and 1F) of the stick, as shown in FIGS. 1D and 1F.

FIGS. 1B, 1C, and 1E illustrate a cross-section, along a plane that is perpendicular to an axis X (108, FIG. 1A) along a stick forming the solid composition, in three example embodiments. In each embodiment, at an end of the stick protruding from the dispenser, herein called the “top” 103 of the composition, along the width (e.g., 120, in FIG. 1B) of the stick, the depth (e.g., 122) of the first portion is gradually decreasing from a larger depth 124 to a smaller depth 126. In these examples, the gradual decrease is shown as linear and monotonic. The smallest depth is illustrated as a point, but can be rounded or square or other shape.

In the examples shown in FIGS. 1A through IF, in a cross-section along a plane that is perpendicular to an axis X along a length of the solid composition, a boundary (see 174 in FIG. 1C) between the first and second blocks has a “V” shape, with two straight lines extending from a point at an angle to each other. Other shapes for the boundary between the first and second blocks can be used. In some implementations, the exterior of the combined blocks has a rectangular-like or oval-like shape with rounded ends and straight edges on long sides, such as shown in FIGS. 1A, 1B, 1C, and 1D. In some implementations, the exterior of the combined blocks has a rectangular shape, such as shown in FIGS. 1E and 1F. Other exterior shapes can be used, such as a rectangular shape with rounded corners, or oval, or elliptical.

In some implementations, the two portions each may have a cross-section that is constant along the entire length of the stick. In some implementations, at least one of the portions may present a cross-section that varies along the length of the stick. The interior part with the darker pigment can have a triangular cross-section, while the exterior part with the lighter pigment can have an oval shape, elliptical shape, rounded-corner rectangular shape, or a shape with straight long sides and rounded ends, or other shape for the cross-section. The interior portion can have other shapes of cross-section such that, when the interior portion and the exterior portion are combined, along the width of the stick an amount of cosmetic from the interior portion is gradually decreased with respect to an amount of cosmetic from the exterior portion.

The oval-shaped stick, with a wider body, for a contour cosmetic product is distinct from normal contours. The wider body allows an individual to cover more of the face in one application.

The dispenser 102 can include a removable cap (not shown) and a bottom section 110. The dispenser 102 may have a device 112 that can be manipulated, e.g., rotated, by an individual, and which pushes the cosmetic composition to make the top of the composition protrude from the dispenser. The dispenser has an interior surface which contains the cosmetic composition within the dispenser.

As an example, the dispenser can use twist-up packaging similar to stick sunscreen or stick deodorant. Such packaging is commercially available from a number of vendors to produce a stick product with a shape with straight long sides and round ends, or other shapes.

Each composition can be prepared using techniques that are conventionally used in the manufacture of cosmetics or dermatology compositions. The cosmetic may be made by extrusion, coextrusion, compacting, or casting one or more compositions to form a stick, a block, or other shape, or into a mold, cup, sheath, or part of a dispenser, regardless of shape. In some implementations, a base formulation is made. Next, distinctively different coloring agents can be added to separate portions of the base formulation. The separate portions can then be combined into a unitary product. In the examples of FIGS. 1A through 1F, the cosmetic comprises two parts A and B which can be formed in a mold. For example, a first step of casting can form part A in part of the dispenser and then a second step of casting can form part B in the remainder of the cup and surrounding the formed part A. As another example, parts A and B can be formed in separate molds, and then combined into a dispenser.

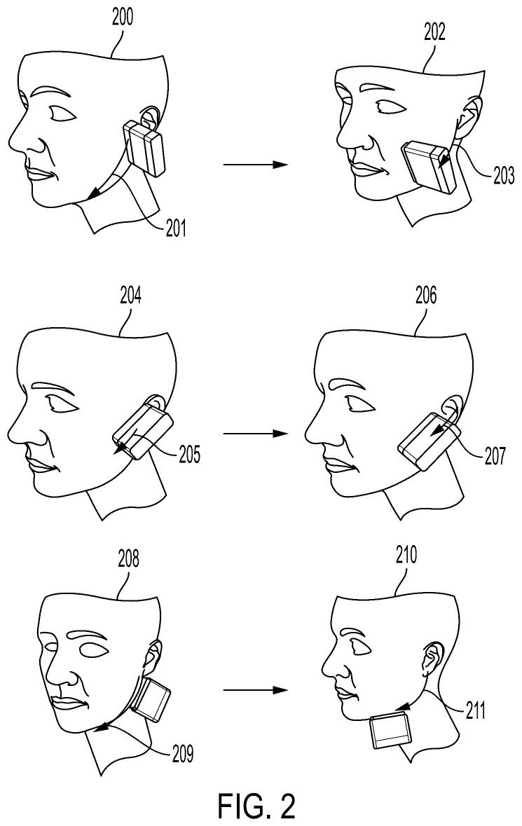

FIG. 2 illustrates several ways in which cosmetics can be applied.

The cosmetic can be applied to the skin in such a manner as to cause the compositions of the blocks to superpose during application. For example, application in a swiping manner superposes the compositions from the different blocks. The swiping action (illustrated by arrows 201, 203, 205, 207, 209, and 211) can be done along the width (e.g., from 200 to 202) or depth (e.g., from 204 to 206, or 208 to 210) of the cosmetic. The cosmetic also can be applied by angling the top of the cosmetic to deposit the composition of each block independently. The cosmetic also can be applied to an applicator or finger, which in turn can be used to apply the cosmetic.

For example, in implementations where the boundary between the first and second blocks has a “V” shape or similarly graded shape, the combination of cosmetics allows for a natural light-to-dark gradient that blends in with the skin when the top of the cosmetic is swiped across the face, such as the cheeks, or lower jaw horizontally due to the ratio of light to dark pigment. As illustrated in FIG. 1B, the combined pigments along line 150 produce a lighter mix 160, while along lines 151 through 154 the combined pigments produce respectively and progressively darker mixes 161 through 164, with the darkest mix 165 occurring along line 155. The cosmetic can also be used vertically to emphasize the jawline, cheekbones, or forehead. The tip or corners (see 140 in FIG. 1B) can also be used to contour defined points like the nose or eyes as well. The stick may be moved in translation so as to leave two colored traces having distinct colors resulting from deposits of the compositions of the different parts.

In some implementations, the contour formula is more sheer than a typical stick contour, as it is not meant to be blended out with a brush or sponge. In the examples shown in the Figures, using FIGS. 1C through 1F, as an example implementation, a sheer contour balm stick is illustrated with a darker shade (at 170) is, and a stick moisturizer with no pigment is illustrated with a lighter shade (at 172). The lighter shade may be colorless or translucent. The darker color should be a few shades darker than the lighter color. The lighter color helps blend out the darker color by mixing. A colorless component, such as this colorless moisturizer, acts as a color ‘dilutant’. In some implementations, such a contour product can have, for example, a matte or natural finish.

The cosmetic may include a cosmetic for application to the face, including but not limited to the cheeks, forehead, or nose, e.g., a contour, a blush, a foundation, a bronzer, or a makeup base, or to the lips, e.g. lip gloss, a lipstick, or a lip balm, or around the eyes, e.g., an eyeliner, or an eyeshadow, or a cosmetic for making up the body or the hair.

Some examples of cool or neutral-toned brown pigments that can be used include, but are not limited to:

| | Pantone | RGB | HEX |
|---|---|---|---|
| 1 | 1R01 SP | 194, 172, 162 | C2ACA2 |
| 2 | 2R04 SP | 197, 161, 146 | C5A192 |
| 3 | 3Y06 SP | 186, 153, 130 | BA9982 |
| 4 | 4Y08 SP | 173, 139, 111 | AD8B6F |
| 5 | 4Y11 SP | 143, 112, 84 | 8F7054 |
| 6 | 4R11 SP | 152, 107, 91 | 986B5B |
| 7 | 2Y14 SP | 109, 80, 57 | 6D5039 |
| 8 | 1Y14.5 SP | 100, 73, 60 | 64493C |
| 9 | 2R15 SP | 81, 66, 57 | 515239 |

Some example combinations of shades are listed in the following table. A kit of two or more different combinations also can be provided.

| Paired | Example | Paired | Example |
|---|---|---|---|
| Shade 1 | pigment | Shade 2 | pigment |
| (“Lighter shade”) | (Pantone) | (“Darker shade”) | (Pantone) |
| Translucent | None | Light | 1R01 SP |
| Light | 2R04 SP | Light Medium | 3Y06 SP |
| Light medium | 4Y11 SP | Medium Deep | 2Y14 SP |
| Medium | 4R11 SP | Deep | 2R15 SP |

In some implementations, the cosmetic includes one or more of the following ingredients. This list of ingredients is neither exhaustive nor limiting. Other ingredients not listed here can be used to make cosmetics in accordance with this specification. Suitable ingredients include, but are not limited to: Polymethyl Methacrylate, Octyldodecanol, Polymethylsilsesquioxane, Pentaerythrityl Tetra-Di-T-Butyl Hydroxyhydrocinnamate, titanium dioxide, Iron oxides, C12-15 Alkyl Benzoate, Wax (Synthetic, Helianthus Annuus (Sunflower) seed, Citrus Aurantium Dulcis (Orange) Peel, etc.), Silica, Tocopheryl Acetate, Caprylic/Capric Triglyceride, Dimethicone, Triethoxycaprylylsilane, and Fluorphlogopite.

The cosmetics are preferably non-comedogenic and problem-skin friendly. For example, there should be no comedogenic/pore-clogging ingredients like essential oils, beeswax, cocoa butter, coconut oil, lanolin, stearic acid, dimethicone, and other ingredients. For “clean beauty” compliance, the following ingredients are further examples (not an exhaustive list) of ingredients not to be used: No Acrylates, Aluminum Salts, Animal Musks/Fats/Oils, Benzophenones and related compounds, Butoxyethanol, Butylated Hydroxyanisole (BHA), Butylated hydroxytoluene (BHT) under 0.1%, Carbon Black (D&C Black No. 2), Coal Tar, Ethanolamine Compounds, Formaldehyde and Formaldehyde Releasing Agents, Hydroquinone, Lead and Lead Acetate, Mercury+Mercury Compounds (Thiomersal), 2-Methoxyethanol, Methyl Cellosolve, Methylchloroisothiazolinone and Methylisothiazolinone, Mineral Oil, Nanomaterials (restrictions for specific nanomaterials only), Oxybenzone, Parabens, Petrolatum and Paraffin [should this say paraffin wax/oil, or just be left alone as paraffin] (USP grade only), Phenoxyethanol under 1%, Phthalates, Plastic Microbeads, Polyacrylamide & Acrylamide, Resorcinol, Retinyl Palmitate, Styrene, Sulfates, Talc (no detection of asbestos), Toluene, Triclosan and Triclocarban, Undisclosed Synthetic Fragrance (under 1% and formulated without Acetaldehyde, Acetone, Acetonitrile, Benzalkonium chloride, BPA, EDTA, Methylene chloride, PTFE, PFOA), 1,4-Dioxane (ingredients must comply with impurity thresholds), Octinoxate, PFAS compounds, Nitromusks and Polycyclic Musks, Ethoxylated Ingredients (including PEGS or polyethylene glycol, and must meet contamination limits for 1,4-Dioxane and ethylene oxide), Cyclic Silicones, EDTA and derivatives (allowed if no technical substitute under 0.2%).

It should be understood that the subject matter defined in the appended claims is not necessarily limited to the specific implementations described above. The specific implementations described above are disclosed as examples only.

What is claimed is:

1. A cosmetics product, comprising:
a solid cosmetic composition forming a stick and including, in a cosmetically-acceptable medium:
at least one first pigment suspended in a first cosmetic composition; and
a second cosmetic composition which is differentiated from the first cosmetic composition by one or more of color, pigment, or finish,
wherein along a width of a top of the stick, a depth of the first cosmetic composition gradually decreases from a larger depth to a smaller depth.

2. The cosmetics product of claim 1, wherein in a cross-section along a plane that is perpendicular to an axis along a length of the solid composition, a boundary between the first cosmetic composition and the second cosmetic composition has a “V” shape, with two straight lines extending from a point at an angle to each other.

3. The cosmetics product of claim 1, wherein the gradual decrease is linear and monotonic.

4. The cosmetics product of claim 1 wherein, in a cross-section along a plane that is perpendicular to an axis along a length of the solid cosmetic composition, a boundary between the first cosmetic composition and the second cosmetic composition has, at the smaller depth of the first cosmetic composition, a rounded shape.

5. The cosmetics product of claim 1, wherein the second cosmetic composition is colorless.

6. A cosmetic product, comprising:
a solid cosmetic composition forming a stick and including:
at least a first cosmetic composition containing, in a cosmetically-acceptable medium, a first pigment, and
a second cosmetic composition containing, in a cosmetically-acceptable medium, a second pigment different from the first pigment,
wherein along a width of a top of the stick, a depth of the first cosmetic composition gradually decreases from a larger depth to a smaller depth.

7. The cosmetics product of claim 6, wherein in a cross-section along a plane that is perpendicular to an axis along a length of the solid cosmetic composition, a boundary between the first cosmetic composition and the second cosmetic composition has a “V” shape, with two straight lines extending from a point at an angle to each other.

8. The cosmetics product of claim 6, wherein the gradual decrease is linear and monotonic.

9. The cosmetics product of claim 6, wherein, in a cross-section along a plane that is perpendicular to an axis along a length of the solid cosmetic composition, a boundary between the first cosmetic composition and the second cosmetic composition has, at the smaller depth of the first cosmetic composition, a rounded shape.

10. The cosmetics product of claim 6, wherein the second cosmetic composition is colorless.

11. A cosmetics product, comprising:
a dispenser; and
a solid cosmetic composition forming a stick and housed in the dispenser, and wherein the solid cosmetic composition has a top protruding from the dispenser, and wherein the solid cosmetic composition includes, in a cosmetically-acceptable medium:
at least one first pigment suspended in a first cosmetic composition, and
a second cosmetic composition which is differentiated from the first cosmetic composition by one or more of color, pigment, or finish,
wherein along a width of the top of the stick, a first amount of the first cosmetic composition is gradually decreased with respect to a second amount of the second cosmetic composition.

12. The cosmetics product of claim 11, wherein in a cross-section along a plane that is perpendicular to an axis along a length of the solid cosmetic composition, a boundary between the first cosmetic composition and the second cosmetic composition has a “V” shape, with two straight lines extending from a point at an angle to each other.

13. The cosmetics product of claim 11, wherein the gradual decrease is linear and monotonic.

14. The cosmetics product of claim 11, wherein, in a cross-section along a plane that is perpendicular to an axis along a length of the solid cosmetic composition, a boundary between the first cosmetic composition and the second cosmetic composition has, at the smaller depth of the first cosmetic composition, a rounded shape.

15. The cosmetics product of claim 11, wherein the second cosmetic composition is colorless.
